(19) United States
(12) Patent Application Publication
(10) Pub. No.: US 2026/0306429 A1
(43) Pub. Date: Oct. 8, 2026

(54) COMPOSITION FOR REDUCING METHANE PRODUCTION AND IMPROVING NITROGEN UTILIZATION EFFICIENCY OF RUMINANT ANIMALS COMPRISING HALOACETAMIDE AS ACTIVE INGREDIENT

(71) Applicant: CHUNGANG UNIVERSITY INDUSTRY ACADEMIC COOPERATION FOUNDATION, Seoul (KR)

(72) Inventors: Tansol PARK, Anseong-si (KR); Chaemin YU, Anseong-si (KR)

(21) Appl. No.: 19/457,002

(22) Filed: Jan. 22, 2026

Related U.S. Application Data
(63) Continuation of application No. PCT/KR2025/099112, filed on Jan. 21, 2025.

(30) Foreign Application Priority Data
Feb. 8, 2024 (KR) 10-2024-0019771
Jul. 18, 2024 (KR) 10-2024-0094865

(51) Int. Cl. A61K 31/16; A61P 1/00

(52) U.S. Cl. CPC A61K 31/16; A61P 1/00

(57) ABSTRACT

The present invention relates to a composition for reducing methane production and improving nitrogen utilization efficiency of ruminant animals, comprising haloacetamide as an active ingredient. The composition for reducing methane production and improving nitrogen utilization efficiency of ruminant animals according to the present invention not only has excellent effects of reducing methane production and improving nitrogen utilization efficiency, but is also characterized by preventing a decrease in feed digestibility in a methane reduction environment. The composition for reducing methane production and improving nitrogen utilization efficiency of ruminant animals according to the present invention is expected to contribute to the commercialization of feed additives capable of significantly alleviating environmental pollution.

### TECHNICAL FIELD

The present invention relates to a composition for reducing methane production and improving nitrogen utilization efficiency in ruminants, comprising haloacetamide as an active ingredient.

### BACKGROUND ART

Methane (CH₄) is the second most abundant greenhouse gas after carbon dioxide (CO₂), accounting for 16% of total greenhouse gas emissions. The potential global warming effect of methane is 28 times greater than that of carbon dioxide. Moreover, methane emissions from ruminants account for 13% to 19% of global methane emissions. Therefore, since ruminant feeding is a major factor that exacerbates global warming, the reduction of methane emissions from ruminants can decrease the rate of global warming, which is crucial for reducing global greenhouse gas emissions. Furthermore, methane emissions contribute to energy loss during the rearing period of ruminants. On average, approximately 8% to 12% of the digested energy from feed is wasted in the form of methane emissions.

Methane is produced in the foregut (rumen) of ruminants by methanogens, which are a subgroup of the Archaea. During normal rumen function, plant materials are degraded by fiber-degrading microorganisms and fermented to produce primarily volatile fatty acids, ammonia, hydrogen, and carbon dioxide. Rumen methanogens primarily use hydrogen to reduce carbon dioxide to methane through a series of reactions coupled with ATP synthesis.

Various attempts have been made to inhibit the activity of methanogens in the rumen, but most have failed or achieved only limited success due to low efficiency, poor selectivity, toxicity of compounds to the host, or increased resistance to anti-methanogen compounds. Therefore, there is an urgent need for methods and for compositions that are effective, selective, and safe for inhibiting methanogens in ruminants.

Representative conventional methane-reducing agents include 3-nitrooxypropanol (3-NOP) disclosed in European Patent No. 2654455 (Applicant: DSM) and PCT International Publication No. WO2020/212348 (Applicant: DSM); freeze-dried Asparagopsis feeds (a red seaweed belonging to the Rhodophyta) disclosed in U.S. Pat. Nos. 9,980,995, 10,881,697, and 11,883,449; nitrates; essential-oil mixtures; saponins; and tannins. 3-NOP has a methane-reducing rate of approximately 20% to 40%, but it has issues related to residues in the animal body and safety. Freeze-dried Asparagopsis (particularly Asparagopsis taxiformis) feed has poor palatability for ruminants and also presents toxicity issues (for example, bromoform contained in Asparagopsis taxiformis is a carcinogenic substance). Nitrates pose toxicity issues due to nitrites generated during the reduction process. Essential-oil mixtures exhibit low methane-reducing efficiency, and saponins not only have toxicity in ruminants but also show low methane-reducing efficiency. Tannins, when administered at high concentrations, not only reduce feed intake and dry matter digestibility but also have low methane-reducing efficiency.

Meanwhile, ruminants exhibit a lower nitrogen utilization efficiency (approximately 15% to 40%) compared to other domestic animals. As a result, a substantial portion of the crude protein supplied through feed is excreted as manure, which may adversely affect the environment. Therefore, there is a need to develop methane-reducing methods that can be practically applied to ruminants and that can improve the nitrogen utilization efficiency of ruminants. Protease inhibitors have been reported to exhibit significant effects on in vitro rumen fermentation and to be effective in inhibiting rumen microbial communities, particularly rumen protozoa.

As a result of extensive research, the inventors of the present invention have completed an invention relating to a composition for reducing methane production and improving nitrogen utilization efficiency in ruminants, and it is expected that this will contribute to the commercialization of a feed additive that can remarkably mitigate environmental pollution.

### Technical Problem

An object the present invention is to provide a composition for reducing methane production and improving nitrogen utilization efficiency in ruminants, comprising haloacetamide as an active ingredient.

### Technical Solution

The present invention provides a composition for reducing methane production and improving nitrogen utilization efficiency in ruminants, comprising haloacetamide as an active ingredient.

In the composition for reducing methane production and improving nitrogen utilization efficiency in ruminants according to the present invention, the haloacetamide is preferably chloroacetamide, iodoacetamide, or a mixture of chloroacetamide and iodoacetamide.

In the composition for reducing methane production and improving nitrogen utilization efficiency in ruminants according to the present invention, the haloacetamide reduces methane production in ruminants by significantly decreasing the copy number of 16S rRNA gene of methanogens.

The mechanisms of action for reducing methane production include: 1) the electrophilic attack of a halogen compound on the nickel (Ni)-containing coenzyme F₄₃₀ (a tetrapyrrole), which functions as a prosthetic group (see FIG. 8); 2) the inhibition of methyl group transfer to coenzyme M (see FIG. 9); and 3) the inhibition of heterodisulfide bond formation between methyl-coenzyme M and coenzyme B (see FIG. 10).

In the composition for reducing methane production and improving nitrogen utilization efficiency in ruminants according to the present invention, the haloacetamide improves nitrogen utilization efficiency and prevents a decrease in feed digestibility in a methane-reducing environment by increasing Fibrobacter succinogenes, Ruminobacter amylophilus, and Succinivibrio dextrinosolvens. Fibrobacter succinogenes, Ruminobacter amylophilus, and Succinivibrio dextrinosolvens are the main taxonomic drivers of haloacetamide-related functions associated with various metabolic activities and microbial protein synthesis within the rumen microbiota. This can account for the increased nitrogen utilization efficiency and the feed digestibility comparable to that of the control group (the haloacetamide-free group) observed in the haloacetamide-treated ruminal microbiome.

Moreover, in the composition for reducing methane production and improving nitrogen utilization efficiency in ruminants according to the present invention, the chloroacetamide increases bacteria belonging to the genera Fibrobacter, Anaeroplasma, Treponema, and Succinivibrio, and the species Ruminococcus albus.

Furthermore, in the composition for reducing methane production and improving nitrogen utilization efficiency in ruminants according to the present invention, the iodoacetamide increases bacteria belonging to the genera Fibrobacter, Pseudobutyrivibrio, and Treponema, and the species Ruminococcus flavefaciens.

In addition, in the composition for reducing methane production and improving nitrogen utilization efficiency in ruminants according to the present invention, the mixture of chloroacetamide and iodoacetamide decreases bacteria belonging to the genera Saccharofermentans and Limimorpha, and the species Fibrobacter succinogenes, Prevotella ruminicola, and Anaerovibrio lipolyticus.

The present invention provides a feed additive comprising the composition for reducing methane production and improving nitrogen utilization efficiency in ruminants.

### Technical Effects

The composition for reducing methane production and improving nitrogen utilization efficiency in ruminants according to the present invention provides excellent effects in reducing methane production and improving nitrogen utilization efficiency, and also prevents a decrease in feed digestibility in a methane-reducing environment.

It is expected that the composition for reducing methane production and improving nitrogen utilization efficiency in ruminants according to the present invention will contribute to the commercialization of a feed additive that can remarkably mitigate environmental pollution.

### BRIEF DESCRIPTION OF THE DRAWINGS

FIG. 1 shows non-metric multi-dimensional scaling (NMDS) plots based on the Bray-Curtis distance matrix, together with pairwise Q-values indicating statistical significance between groups based on the Bonferroni correction.

FIG. 2 shows the characteristics of the taxonomic drivers of MetaCyc pathways abundant in the haloacetamide-associated rumen microbiome, analyzed using Fish Taco.

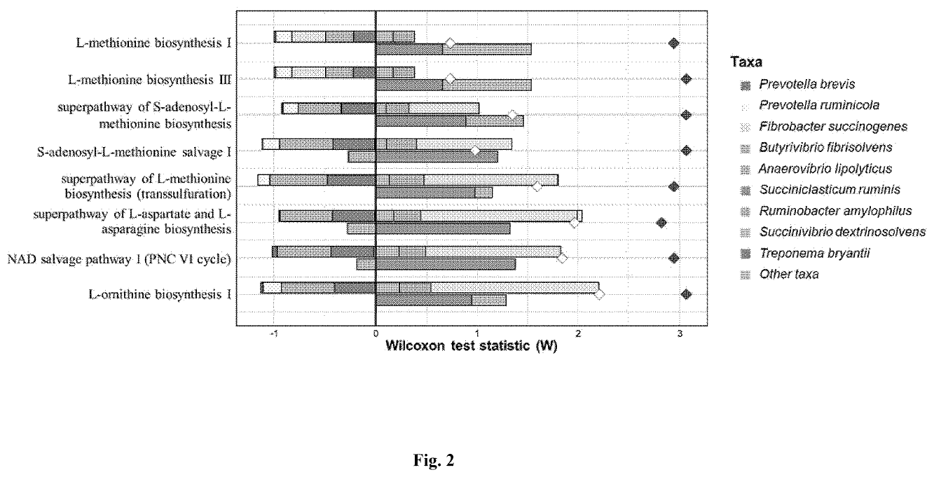

FIG. 3 shows the genera and species of microorganisms that are differentially abundant in response to haloacetamide treatment.

FIG. 4 illustrates the mechanism of action for reducing methane production, showing the electrophilic attack of a halogen compound on the nickel (Ni)-containing coenzyme F₄₃₀ (a tetrapyrrole), which functions as a prosthetic group.

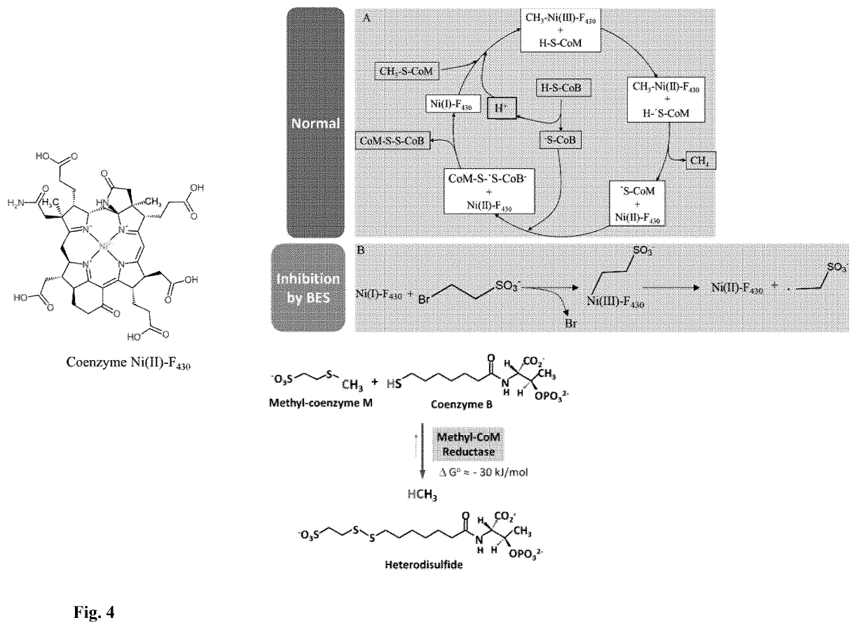

FIG. 5 illustrates the mechanism of action for reducing methane production, showing the inhibition of methyl group transfer to coenzyme M.

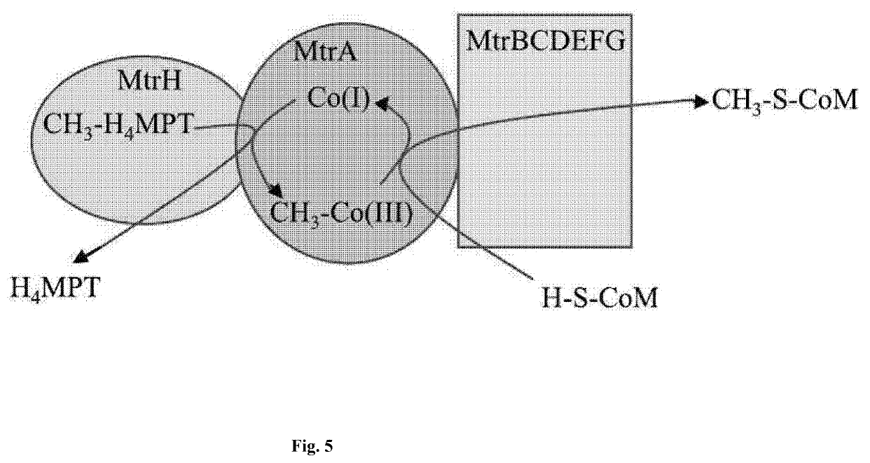

FIG. 6 shows illustrates the mechanism of action for reducing methane production, showing the inhibition of heterodisulfide bond formation between methyl-coenzyme M and coenzyme B.

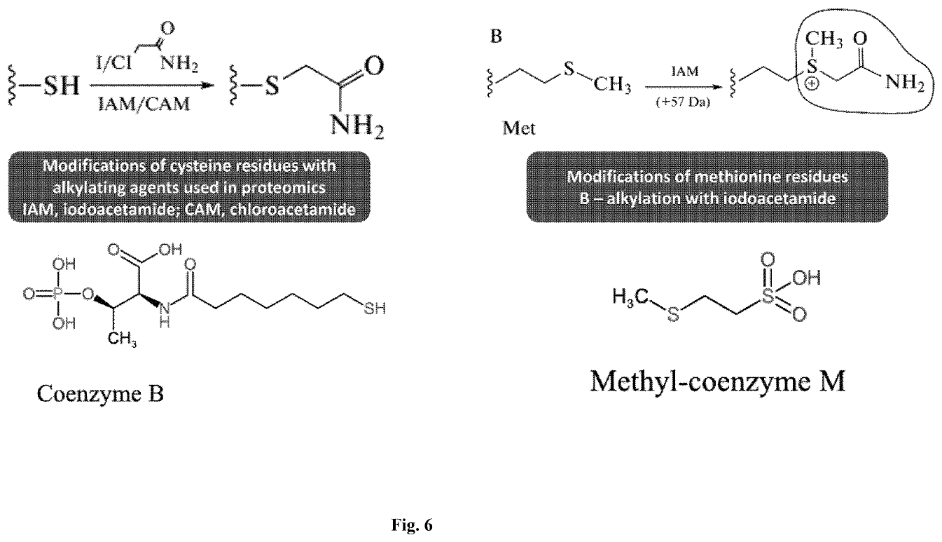

### DETAILED DESCRIPTION

The following examples are provided to illustrate the present invention in greater detail. However, the scope of the present invention should not be construed as being limited by the examples set forth below.

### Experimental Methods

### Experimental Design and In Vitro Incubation of Rumen Fluid

Rumen fluid was sampled from Hanwoo cows at the Nonghyup Research Farm in Anseong, Gyeonggi Province, and mixed with buffer at a ratio of 1:2 within 1 hour of sampling. Oat hay was used as the forage, and pelleted feed for the middle fattening period was used as the concentrate. After grinding, the forage and concentrate were mixed at a ratio of 1:1 after passing through a 1-mm screen. 50 ml of culture medium was dispensed into 0.5 g of dry matter, and the control group and the five experimental groups (acrylamide, chloroacetamide, glycolamide, iodoacetamide, and propionamide) were each incubated for 24 hours with five replicates. The control group was incubated in 50 mL of culture medium, whereas the experimental groups were incubated in 49.9 mL of culture medium plus 0.1 mL of deionized water. For the five experimental groups, the supplementation level of each amide was set to 0.2 mmol/L (final concentration), and the amounts added were 1.4216 mg/g for acrylamide, 1.8702 mg/g for 2-chloroacetamide, 1.4618 mg/g for propionamide, 1.5014 mg/g for glycolamide, and 3.6992 mg/g for iodoacetamide.

### Sample Collection and Analysis

Gas pressure was measured at 0, 3, 6, 12, and 24 hours (gas collection intervals), and the gas released during each measurement was collected to determine the total gas production and methane production up to the final 24 hours. After completing the 24-hour incubation, pH, ammonia nitrogen (NH₃—N), dry matter digestibility, neutral detergent fiber digestibility, volatile fatty acids (VFA), and the microbiome were analyzed.

DNA Extraction, qPCR, and Metataxonomic Analysis

Metagenomic DNA was extracted using the RBB+C method, followed by qPCR analysis and microbiome analysis. Microbial functions were predicted using PICRUSt2. Taxonomic drivers of differentially enriched MetaCyc pathways were identified using Fish Taco (Functional Shifts' Taxonomic Contributor).

### Statistical Analysis

Data conforming to normal distribution and homogeneity of variance were analyzed using the GLIMMIX procedure of SAS and Tukey's HSD test. Data that did not conform to normal distribution or homogeneity of variance were analyzed using the non-parametric Kruskal-Wallis H test, followed by Dunn's post hoc test in R to identify pairwise differences between groups after confirming overall significance by ANOVA. Differences in the overall bacteriota and their functional profiles were analyzed using PERMANOVA. Differentially abundant microbial biomarkers were identified using multivariable association analysis with the MaAsLin2 R package, based on relatively abundant bacterial taxa and predicted microbial functions.

### Experimental Results

### Rumen Fermentation Characteristics

In vitro digestibility and rumen fermentation characteristics are presented in Table 1 below.

TABLE 1

| Item | CON | AA | CLA | GA | IA | PA | SEM | P-value |
|---|---|---|---|---|---|---|---|---|
| DMD (%) | 78.60^{AB} | 78.14^{AB} | 77.82^{AB} | 79.40^{A} | 76.18^{B} | 78.34^{AB} | 1.38 | 0.0290 |
| NDFD (%) | 41.77^{B} | 42.04^{B} | 43.61^{B} | 40.82^{B} | 47.83^{A} | 41.80^{B} | 1.76 | <.0001 |
| pH | 6.19 | 6.21 | 6.19 | 6.22 | 6.19 | 6.22 | 0.02 | 0.0723 |
| NH₃—N (mg/dL) | 15.95^{A} | 16.61^{A} | 13.90^{B} | 14.76^{AB} | 13.16^{B} | 16.50^{A} | 1.01 | <.0001 |
| TGP (mL) | 146.69^{A} | 145.83^{A} | 134.49^{B} | 141.74^{A} | 129.37^{B} | 143.63^{A} | 2.71 | <.0001 |
| CH₄ (mL) | 11.54^{A} | 11.35^{A} | 4.17^{C} | 10.65^{B} | 0.23^{D} | 11.09^{AB} | 0.36 | <.0001 |
| CH₄ (mL/g dDM) | 29.37^{A} | 29.06^{A} | 10.71^{C} | 26.85^{B} | 0.60^{D} | 28.32^{AB} | 1.09 | <.0001 |
| Total VFA (mM) | 68.4^{AB} | 70.82^{A} | 63.14^{AB} | 61.27^{AB} | 59.25^{B} | 63.20^{AB} | 6.17 | 0.0259 |
| Acetate (%) | 61.39^{A} | 61.85^{A} | 55.96^{B} | 60.74^{A} | 52.09^{C} | 60.97^{A} | 0.92 | <.0001 |
| Propionate (%) | 20.37^{C} | 20.42^{C} | 23.89^{B} | 20.83^{C} | 27.56^{A} | 20.64^{C} | 0.29 | <.0001 |
| Isobutyrate (%) | 0.93 | 0.88 | 0.80 | 0.93 | 0.76 | 0.92 | 0.11 | 0.0939 |
| Butyrate (%) | 14.15^{B} | 13.87^{B} | 16.30^{A} | 14.30^{B} | 16.51^{A} | 14.31^{B} | 0.46 | <.0001 |
| Isovalerate (%) | 1.31^{AB} | 1.23^{ABC} | 1.09^{BC} | 1.32^{A} | 1.02^{C} | 1.30^{AB} | 0.11 | 0.0070 |
| Valerate (%) | 1.85^{AB} | 1.76^{B} | 1.95^{AB} | 1.88^{AB} | 2.06^{A} | 1.86^{AB} | 0.14 | 0.0454 |
| A:P ratio | 3.02^{A} | 3.03^{A} | 2.34^{B} | 2.92^{A} | 1.89^{C} | 2.96^{A} | 0.07 | <.0001 |

In the table above, the terms are defined as follows:
CON: control
AA: acrylamide
CLA: chloroacetamide
GA: glycolamide
IA: iodoacetamide
PA: propionamide
DMD: dry matter digestibility
NDFD: neutral detergent fiber digestibility
NH₃—N: ammonia nitrogen
TGP: total gas production
CH₄ (mL/g dDM): methane production (mL) per gram (g) of degraded dry matter
Total VFA: total volatile fatty acids
A:P ratio: acetate:propionate ratio
SEM: standard error of the mean

As shown in Table 1 above, haloacetamides such as chloroacetamide and iodoacetamide were confirmed to exhibit excellent methane-reducing effects.

### Volatile Fatty Acid (VFA) Profiles

The volatile fatty acid (VA) profiles for the control group and the five experimental groups (acrylamide, chloroacetamide, glycolamide, iodoacetamide, and propionamide) are presented in Table 2 below.

TABLE 2

| Item | CON | AA | CLA | GA | IA | PA | Pooled SEM | P-value |
|---|---|---|---|---|---|---|---|---|
| Concentration (mM) | | | | | | | | |
| Total VFA | 68.40^{AB} | 70.82^{A} | 63.14^{AB} | 61.27^{AB} | 59.25^{B} | 63.20^{AB} | 6.17 | 0.0259 |
| Acetate | 42.05^{AB} | 43.81^{A} | 35.41^{BC} | 37.24^{ABC} | 29.92^{C} | 38.55^{AB} | 4.07 | 0.0003 |
| Propionate | 13.90^{AB} | 14.45^{AB} | 15.07^{AB} | 12.77^{B} | 15.80^{A} | 13.04^{B} | 1.33 | 0.0099 |
| Isobutyrate | 0.63^{A} | 0.62^{A} | 0.50^{BC} | 0.57^{AB} | 0.43^{C} | 0.58^{AB} | 0.05 | <.0001 |
| Butyrate | 9.66^{AB} | 9.82^{AB} | 10.25^{A} | 8.74^{B} | 9.45^{AB} | 9.04^{AB} | 0.76 | 0.0552 |
| Isovalerate | 0.89^{A} | 0.87^{A} | 0.68^{B} | 0.80^{A} | 0.58^{B} | 0.82^{A} | 0.05 | <.0001 |
| Valerate | 1.26 | 1.24 | 1.22 | 1.15 | 1.18 | 1.17 | 0.06 | 0.0656 |
| Composition (%) | | | | | | | | |
| Acetate | 61.39^{A} | 61.85^{A} | 55.96^{B} | 60.74^{A} | 52.09^{C} | 60.97^{A} | 0.92 | <.0001 |
| Propionate | 20.37^{C} | 20.42^{C} | 23.89^{B} | 20.83^{C} | 27.56^{A} | 20.64^{C} | 0.29 | <.0001 |
| Isobutyrate | 0.93 | 0.88 | 0.80 | 0.93 | 0.76 | 0.92 | 0.11 | 0.0939 |
| Butyrate | 14.15^{B} | 13.87^{B} | 16.30^{A} | 14.30^{B} | 16.51^{A} | 14.31^{B} | 0.46 | <.0001 |
| Isovalerate | 1.31^{AB} | 1.23^{ABC} | 1.09^{BC} | 1.32^{A} | 1.02^{C} | 1.30^{AB} | 0.11 | 0.0070 |
| Valerate | 1.85^{AB} | 1.76^{B} | 1.95^{AB} | 1.88^{AB} | 2.06^{A} | 1.86^{AB} | 0.14 | 0.0454 |
| A:P ratio | 3.02^{A} | 3.03^{A} | 2.34^{B} | 2.92^{A} | 1.89^{C} | 2.96^{A} | 0.07 | <.0001 |

AA: acrylamide,
CLA: chloroacetamide,
GA: glycolamide,
IA: iodoacetamide,
PA: propionamide
** VFA: Volatile fatty acids

### Quantitative Real-Time PCR Data

For the control group and the five experimental groups (acrylamide, chloroacetamide, glycolamide, iodoacetamide, and propionamide), the effects of the amide compounds on the absolute quantification of total bacteria, methanogens, protozoa, and specific cellulolytic bacterial species (Fibrobacter succinogenes, Ruminococcus flavefaciens, Ruminococcus albus, and Prevotella ruminicola) are presented in Table 3 below as quantitative real-time PCR data.

TABLE 3

| Item | CON | AA | CLA | GA | IA | PA | SEM | P-value |
|---|---|---|---|---|---|---|---|---|
| Total bacteria | 10.27^{B} | 10.30^{AB} | 10.28^{B} | 10.25^{B} | 10.38^{A} | 10.27^{B} | 0.04 | 0.0017 |
| Total methanogen | 8.16^{A} | 8.38^{A} | 8.05^{AB} | 8.33^{A} | 7.74^{B} | 8.42^{A} | 0.20 | 0.0002 |
| Total protozoa | 9.74 | 9.78 | 9.77 | 9.69 | 9.68 | 9.79 | 0.07 | 0.0911 |
| Fibrobacter succinogenes | 9.32^{C} | 9.39^{C} | 9.54^{B} | 9.36^{C} | 9.79^{A} | 9.35^{C} | 0.07 | <.0001 |
| Ruminococcus flavefaciens | 8.26^{B} | 8.31^{B} | 8.27^{B} | 8.29^{B} | 8.51^{A} | 8.31^{B} | 0.08 | 0.0003 |
| Ruminococcus albus | 7.73^{B} | 7.77^{B} | 7.96^{A} | 7.71^{B} | 7.83^{AB} | 7.71^{B} | 0.08 | 0.0005 |
| Prevotella ruminicola | 9.43^{C} | 9.44^{C} | 9.58^{B} | 9.42^{C} | 9.72^{A} | 9.45^{C} | 0.04 | <.0001 |

### Alpha-Diversity Measurements

The alpha-diversity measurements for the control group and the five experimental groups (acrylamide, chloroacetamide, glycolamide, iodoacetamide, and propionamide) are presented in Table 4 below.

TABLE 4

| | | | Observed | | |
|---|---|---|---|---|---|
| | Chao1 | Evenness | features | Shannon | Simpson |
| CON | 398.7^{AB} | 0.8923^{AB} | 396.8^{AB} | 7.6638^{A} | 0.9913^{AB} |
| AA | 284.8^{B} | 0.8823^{B} | 284.8^{B} | 7.1896^{B} | 0.9883^{B} |
| CLA | 382.3^{AB} | 0.8847^{AB} | 380.8^{AB} | 7.5719^{AB} | 0.9903^{AB} |
| GA | 436.4^{AB} | 0.8956^{A} | 435.6^{AB} | 7.8466^{A} | 0.9927^{A} |
| IA | 525.3^{A} | 0.8895^{AB} | 520.6^{A} | 8.0215^{A} | 0.9925^{A} |
| PA | 406.2^{AB} | 0.8873^{AB} | 405.6^{AB} | 7.6692^{A} | 0.9913^{AB} |
| Pooled SEM | 66.6559 | 0.0062 | 65.0820 | 0.2412 | 0.0013 |
| P-value | 0.0052 | 0.0269 | 0.0060 | 0.0005 | 0.0067 |

AA: acrylamide,
CLA: chloroacetamide,
GA: glycolamide,
IA: iodoacetamide,
PA: propionamide

### Beta-Diversity Measurements

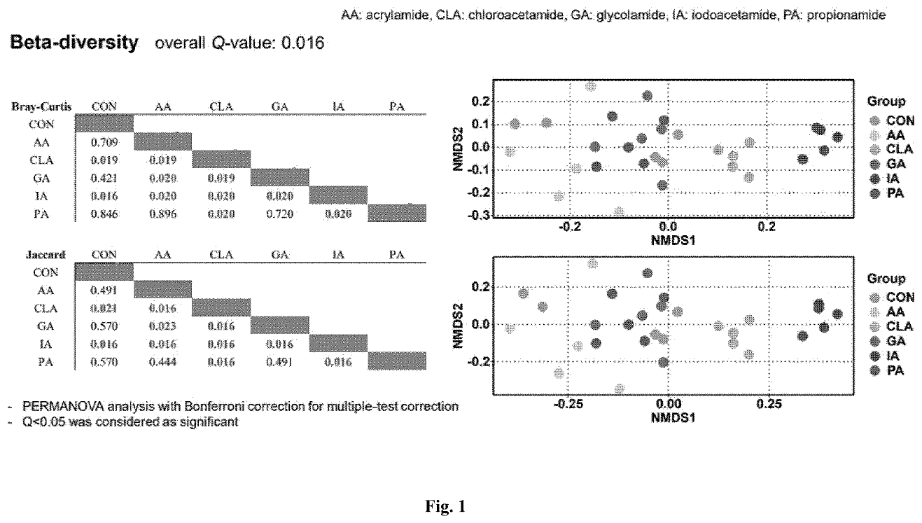

The beta-diversity measurements for the control group and the five experimental groups (acrylamide, chloroacetamide, glycolamide, iodoacetamide, and propionamide) are shown in FIG. 1.

### Differentially Abundant Genera and Species in Response to Haloacetamide Treatment

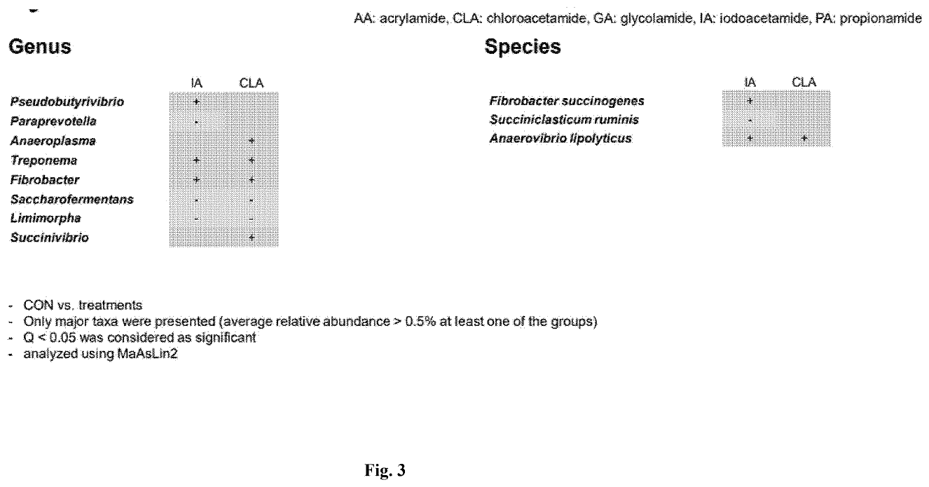

The genera and species that were differentially abundant in response to haloacetamide treatment are shown in FIG. 3.

1. A composition for reducing methane production and improving nitrogen utilization efficiency in ruminants, comprising haloacetamide as an active ingredient.

2. The composition for reducing methane production and improving nitrogen utilization efficiency in ruminants according to claim 1, wherein the haloacetamide is chloroacetamide, iodoacetamide, or a mixture of chloroacetamide and iodoacetamide.

3. The composition for reducing methane production and improving nitrogen utilization efficiency in ruminants according to claim 1, wherein the haloacetamide reduces methane production in ruminants by significantly decreasing the copy number of 16S rRNA gene of methanogens.

4. The composition for reducing methane production and improving nitrogen utilization efficiency in ruminants according to claim 1, wherein the haloacetamide improves nitrogen utilization efficiency and prevents a decrease in feed digestibility in a methane-reducing environment by increasing Fibrobacter succinogenes, Ruminobacter amylophilus, and Succinivibrio dextrinosolvens.

5. The composition for reducing methane production and improving nitrogen utilization efficiency in ruminants according to claim 2, wherein the chloroacetamide increases bacteria belonging to the genera Fibrobacter, Anaeroplasma, Treponema, and Succinivibrio, and the species Ruminococcus albus.

6. The composition for reducing methane production and improving nitrogen utilization efficiency in ruminants according to claim 2, wherein the iodoacetamide increases bacteria belonging to the genera Fibrobacter, Pseudobutyrivibrio, and Treponema, and the species Ruminococcus flavefaciens.

7. The composition for reducing methane production and improving nitrogen utilization efficiency in ruminants according to claim 2, wherein the mixture of chloroacetamide and iodoacetamide decreases bacteria belonging to the genera Saccharofermentans and Limimorpha, and the species Fibrobacter succinogenes, Prevotella ruminicola, and Anaerovibrio lipolyticus.

8. A feed additive comprising the composition for reducing methane production and improving nitrogen utilization efficiency in ruminants according to claim 1.
